(19) United States
(12) Patent Application Publication
(10) Pub. No.: US 2024/0156365 A1
(43) Pub. Date: May 16, 2024

(54) AN APPARATUS AND A METHOD FOR MEASURING FLOW RESISTANCES OF BLOOD VESSELS

(71) Applicant: TURUN YLIOPISTO, Turun yliopisto (FI)

(72) Inventors: Matti KAISTI, Turku (FI); Tuukka PANULA, Turku (FI); Jukka-Pekka SIRKIÄ, Turku (FI)

(21) Appl. No.: 18/548,788

(22) PCT Filed: Feb. 23, 2022
(86) PCT No.: PCT/FI2022/050117

§ 371 Date: Sep. 1, 2023

(30) Foreign Application Priority Data
Mar. 4, 2021 (FI) 20215239

(51) Int. Cl. A61B 5/026; A61B 5/02; A61B 5/022; A61B 5/024

(52) U.S. Cl. CPC A61B 5/0261; A61B 5/02007; A61B 5/02241; A61B 5/02416

(57) ABSTRACT

An apparatus for measuring flow resistances of blood vessels includes a photoplethysmography sensor for emitting electromagnetic radiation with different wavelengths to the blood vessels, a pressure instrument for applying controllable mechanical pressure on the blood vessels, and a control system for estimating compliances of the blood vessels based on electromagnetic radiation reflected off the blood vessels. Shorter wavelengths of the electromagnetic radiation are reflected off smaller blood vessels than longer wavelengths. The control system optimizes resistor values of a circuit model to minimize differences between waveforms of node voltages of the circuit model and waveforms of measured blood pressures prevailing in blood vessels reflecting off different wavelengths. Capacitor values of the circuit model are based on the estimated compliances of the blood vessels, and the optimized resistor values of the circuit model are indicative of the flow resistances of the blood vessels.

### FIELD OF THE DISCLOSURE

The disclosure relates to an apparatus and a method for measuring flow resistances of blood vessels. Furthermore, the disclosure relates to a computer program for measuring flow resistances of blood vessels.

### BACKGROUND

In many cases, quantitative assessment of vascular pathology and its progression involves measurement and estimation of different quantitative measures such as for example: blood pressure, a blood vessel cross sectional area, a blood vessel mean diameter, blood vessel stiffness, and blood vessel flow resistance. For example, abnormal blood vessel stiffness and flow resistance are associated with an increased risk of cardiovascular events such as myocardial infarction and stroke, which are two leading causes of death in the developed world. An increase in arterial stiffness and flow resistance may increase the load of the heart, since the heart needs to perform more work to maintain a required blood flow volume. Over time, this increased workload may cause left ventricular hypertrophy and left ventricular remodelling, which can lead to a heart failure. The increased workload may also be associated with a higher heart rate, a proportionately longer duration of systole, and a reduction of duration of diastole. This decreases the amount of time available for perfusion of cardiac tissue, which mainly occurs during diastole. Thus, a hypertrophic heart, which has a greater oxygen demand, may have a compromised supply of oxygen and nutrients.

Due to the reasons of the kind mentioned above, several techniques have been developed to measure and/or estimate different quantitative measures related to blood vessels. Publication Jukka-Pekka Sirkiä et al.: Multi-Wavelength Photoplethysmography Device for the Measurement of Pulse Transit Time in the Skin Microvasculature, Computing in Cardiology, 2020-09-16 describes a multiwavelength photoplethysmography “MWPPG” device for studying the skin microvasculature. The device utilizes the fact that the penetration depth of light into the skin is depended on the light wavelength. Thus, the device allows to study blood vessels at different depths.

Publication US2017172430 describes a method for cuff-less blood pressure measurement. The method comprises recording a physiological signal and multi-wavelength photoplethysmography “PPG” signals from a predetermined body part, deriving the depth-specific PPG signal reflecting the arterial blood volume with the physiological signal as a reference, calculating the pulse transit time “PTT” from the physiological signal and the derived arterial blood PPG signal, and calculating the blood pressure from the calculated PTT and blood pressure relationship.

Publication US2019336016 describes a device for non-invasive capillary blood pressure measurement. The device comprises a front end in contact with a body to compress and decompress capillaries in tissue, a pressure control module for regulating contact pressure between the front end and the tissue, a pressure transducer coupled to the front end for measuring the contact pressure, a capillary sensing module for detecting capillary pulsations under the contact pressure modulation, and a computing system for running an algorithm to determine capillary pressure based on the capillary pulsations and the contact pressure modulation.

There is, however, still a need for techniques for measuring flow resistances of blood vessels quickly and cost effectively.

### SUMMARY

The following presents a simplified summary in order to provide a basic understanding of some aspects of various invention embodiments. The summary is not an extensive overview of the invention. It is neither intended to identify key or critical elements of the invention nor to delineate the scope of the invention. The following summary merely presents some concepts of the invention in a simplified form as a prelude to a more detailed description of exemplifying embodiments of the invention.

In accordance with the invention, there is provided a new apparatus for measuring flow resistances of blood vessels. An apparatus according to the invention comprises:
a photoplethysmography “PPG” sensor configured to emit electromagnetic radiation to the blood vessels, to receive a part of the electromagnetic radiation reflected off the blood vessels, and to produce a measurement signal indicative of the received part of the electromagnetic radiation so that the electromagnetic radiation has different wavelengths and the measurement signal comprises wavelength-specific component signals indicative of received wavelengths reflected off the blood vessels, where a shorter one of the wavelengths is reflected off smaller ones of the blood vessels than a longer one of the wavelengths,
a pressure instrument configured to produce controllable mechanical pressure applied on the blood vessels, and
a control system configured to find, from each of the wavelength-specific component signals, a portion whose envelope, i.e. a curve outlining extremes of the wavelength-specific component signal, has exponential change, i.e. exponential growth or exponential decrease, with respect to time when the mechanical pressure changes, i.e. decreases or increases, linearly with respect to time, and to produce, for each of the wavelength-specific component signals, an estimate for a coefficient of time related to the exponential change, the coefficients of time being indicative of compliances of the blood vessels reflecting off the different wavelengths.

The control system is configured to optimize resistance values of a circuit model to minimize differences between waveforms of node voltages of the circuit model and measured waveforms of blood pressures being in the blood vessels reflecting off different ones of the wavelengths, wherein capacitor values of the circuit model are based on the above-mentioned compliances of the blood vessels reflecting off the different wavelengths and the optimized resistance values are indicative of the flow resistances of the blood vessels reflecting off the different wavelengths.

The above-mentioned pressure instrument can be for example a device for directing mechanical pressure to a fingertip or a toe, or a device comprising a cuff and a pump system for controlling gas pressure inside the cuff to direct mechanical pressure to an arm.

The photoplethysmography “PPG” sensor can be configured to emit electromagnetic radiation having wavelength in the range from 625 nm to 1000 nm, i.e. red or infrared light, in order to measure the flow resistance of arteries located in the hypodermis, and electromagnetic radiation having wavelength in the range from 565 nm to 590 nm, i.e. yellow light, in order to measure the flow resistance of blood vessels located in an upper portion of the hypodermis, and electromagnetic radiation having wavelength in the range from 500 nm to 565 nm, i.e. green light, in order to measure the flow resistance of arterioles located in the dermis, and electromagnetic radiation having wavelength in the range from 450 nm to 485 nm, i.e. blue light, in order to measure the flow resistance of capillaries located in an upper portion of the dermis.

In accordance with the invention, there is provided also a new method for measuring flow resistances of blood vessels. A method according to the invention comprises:
emitting electromagnetic radiation to the blood vessels so that the electromagnetic radiation has different wavelengths,
receiving a part of the electromagnetic radiation reflected off the blood vessels,
producing a measurement signal indicative of the received part of the electromagnetic radiation so that the measurement signal comprises wavelength-specific component signals indicative of received wavelengths reflected off the blood vessels, where a shorter one of the wavelengths is reflected off smaller ones of the blood vessels than a longer one of the wavelengths,
producing mechanical pressure applied on the blood vessels,
finding, from each of the wavelength-specific component signals, a portion whose envelope has exponential change with respect to time when the mechanical pressure changes linearly with respect to time,
producing, for each of the wavelength-specific component signals, an estimate for a coefficient of time related to the exponential change, the coefficient of time being indicative of compliance of the blood vessels reflecting off the wavelength under consideration, and
optimizing resistance values of a circuit model to minimize differences between waveforms of node voltages of the circuit model and measured waveforms of blood pressures being in the blood vessels reflecting off different ones of the wavelengths, wherein capacitor values of the circuit model are based on the compliances of the blood vessels and the optimized resistance values are indicative of the flow resistances of the blood vessels.

In accordance with the invention, there is provided also a new computer program for measuring flow resistances of blood vessels. A computer program according to the invention comprises computer executable instructions for controlling a programmable processing system to:
control a photoplethysmography sensor to emit electromagnetic radiation to the blood vessels, to receive a part of the electromagnetic radiation reflected off the blood vessels, and to produce a measurement signal indicative of the received part of the electromagnetic radiation so that the electromagnetic radiation has different wavelengths and the measurement signal comprises wavelength-specific component signals indicative of received wavelengths reflected off the blood vessels, where a shorter one of the wavelengths is reflected off smaller ones of the blood vessels than a longer one of the wavelengths,
control a pressure instrument to produce mechanical pressure applied on the blood vessels,
control the programmable processing system to find, from each of the wavelength-specific component signals, a portion whose envelope has exponential change with respect to time when the mechanical pressure changes linearly with respect to time, and produce, for each of the wavelength-specific component signals, an estimate for a coefficient of time related to the exponential change, the coefficient of time being indicative of compliance of the blood vessels reflecting off the wavelength under consideration, and
optimize resistance values of a circuit model to minimize differences between waveforms of node voltages of the circuit model and measured waveforms of blood pressures being in the blood vessels reflecting off different ones of the wavelengths, wherein capacitor values of the circuit model are based on the compliances of the blood vessels and the optimized resistance values are indicative of the flow resistances of the blood vessels.

In accordance with the invention, there is provided also a new computer program product. A computer program product according to the invention comprises a non-volatile computer readable medium, e.g. a compact disc “CD”, encoded with a computer program according to the invention.

Exemplifying and non-limiting embodiments are described in accompanied dependent claims.

Various exemplifying and non-limiting embodiments both as to constructions and to methods of operation, together with additional objects and advantages thereof, will be best understood from the following description of specific exemplifying embodiments when read in conjunction with the accompanying drawings.

The verbs “to comprise” and “to include” are used in this document as open limitations that neither exclude nor require the existence of also un-recited features.

The features recited in the accompanied dependent claims are mutually freely combinable unless otherwise explicitly stated.

Furthermore, it is to be understood that the use of “a” or “an”, i.e. a singular form, throughout this document does not exclude a plurality.

### BRIEF DESCRIPTION OF FIGURES

Exemplifying and non-limiting embodiments and their advantages are explained in greater detail below with reference to the accompanying drawings, in which:

FIG. 1a illustrates an apparatus according to an exemplifying and non-limiting embodiment for measuring flow resistances of blood vessels,

FIG. 1b shows exemplifying graphs illustrating electromagnetic radiations having different wavelengths and reflected off blood vessels of a fingertip as a function of time,

FIG. 1c shows the exemplifying graphs of FIG. 1b converted to a logarithmic vertical scale,

FIG. 1d shows an exemplifying circuit model used for estimating the flow resistances of blood vessels,

FIG. 2 illustrates an apparatus according to an exemplifying and non-limiting embodiment for measuring flow resistances of blood vessels, and

FIG. 3 shows a flowchart of a method according to an exemplifying and non-limiting embodiment for measuring flow resistances of blood vessels.

### DESCRIPTION OF EXEMPLIFYING AND NON-LIMITING EMBODIMENTS

The specific examples provided in the description below should not be construed as limiting the scope and/or the applicability of the appended claims. Lists and groups of examples provided in the description are not exhaustive unless otherwise explicitly stated.

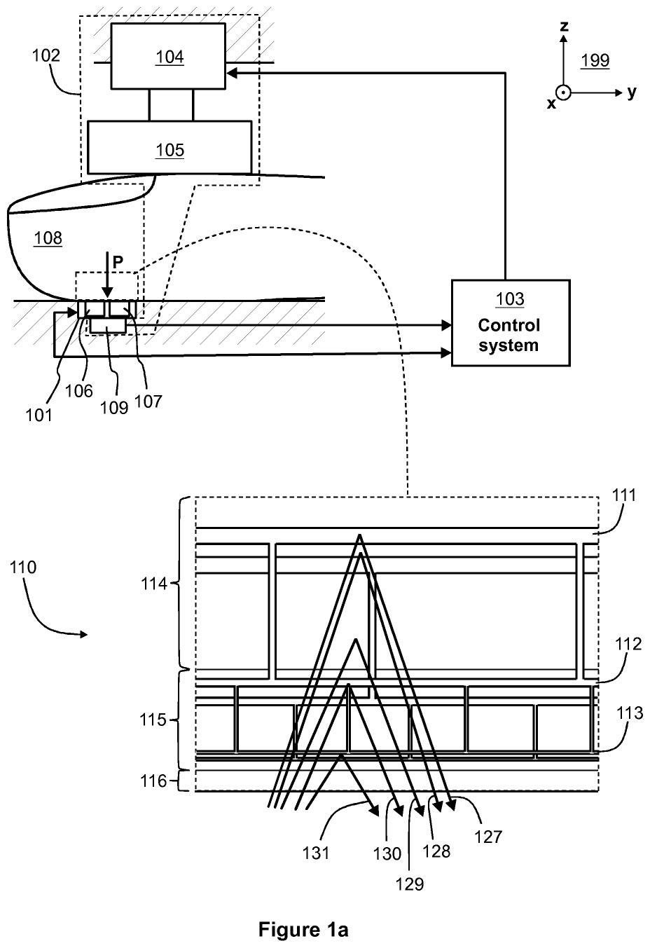

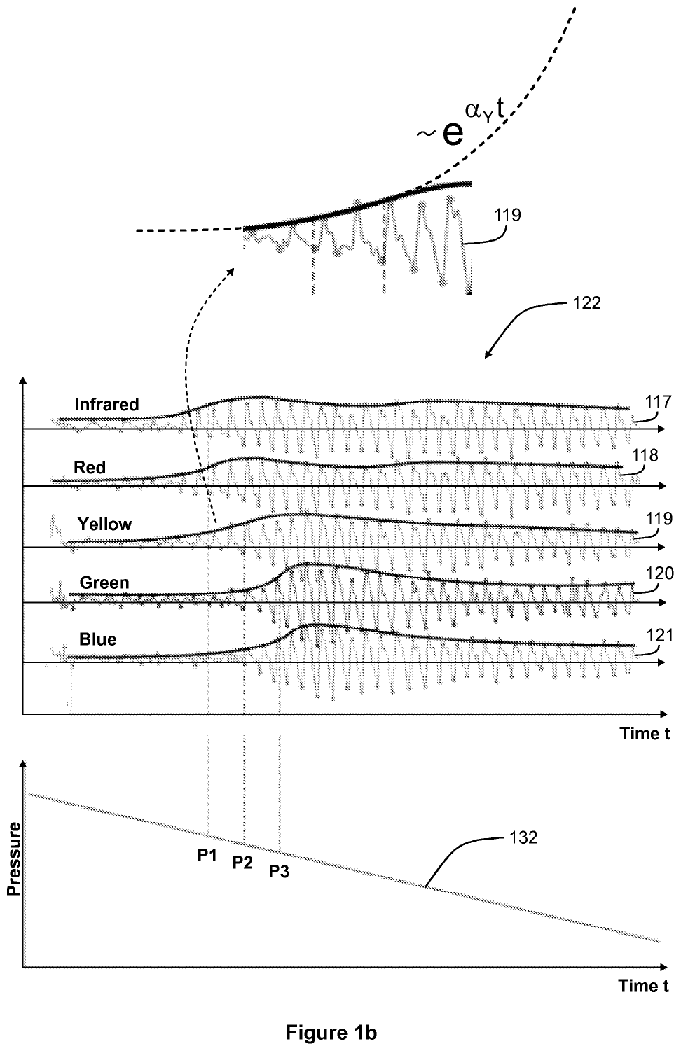

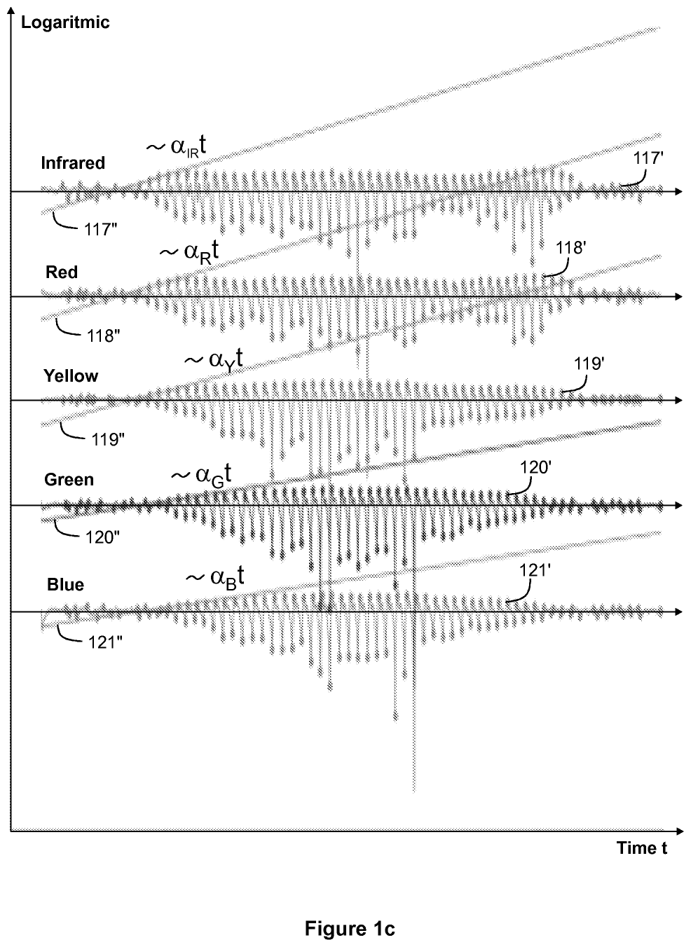

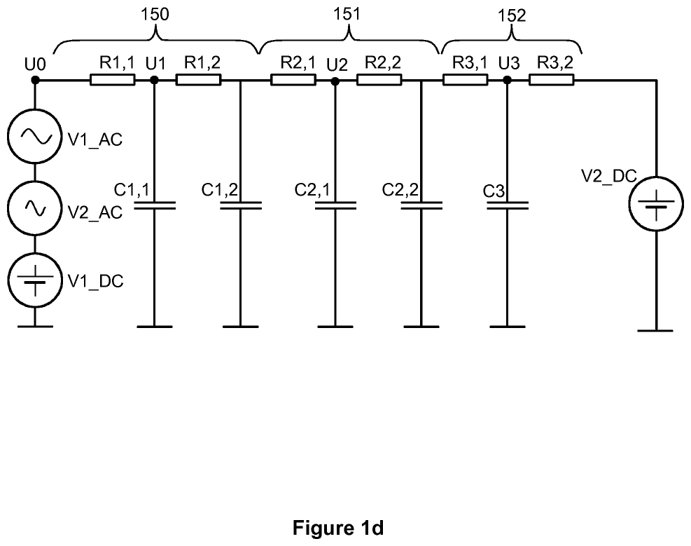

FIG. 1a shows a schematic illustration of an apparatus according to an exemplifying and non-limiting embodiment for measuring flow resistances of blood vessels. The apparatus comprises a photoplethysmography “PPG” sensor 101 for emitting, to a fingertip 108 of an individual, electromagnetic radiation and for receiving a part of the electromagnetic radiation reflected off the blood vessels of the fingertip 108. The PPG sensor 101 comprises a radiation emitter 106 and a photodetector 107. The radiation emitter 106 may comprises e.g. one or more light emitting diodes “LED” and the photodetector 107 may comprise e.g. one or more photodiodes or phototransistors. FIG. 1a shows also a magnified, schematic section view 110 of the fingertip. The section plane is parallel with the yz-plane of a coordinate system 199.

In the exemplifying apparatus illustrated in FIG. 1a, the PPG sensor 101 is configured to emit the electromagnetic radiation so that the electromagnetic radiation contains radiation components with five different wavelengths. In the section view 110, the radiation components are depicted with polyline arrows 127, 128, 129, 130, and 131. The first radiation component 127 can be for example infrared radiation having a wavelength on the range from 700 nm to 1000 nm, the second radiation component 128 can be for example red radiation having a wavelength on the range from 625 nm to 700 nm, the third radiation component 129 can be for example yellow radiation having a wavelength on the range from 565 nm to 590 nm, the fourth radiation component 130 can be for example green radiation having a wavelength on the range from 500 nm to 565 nm, and the fifth radiation component 131 can be for example blue radiation having a wavelength on the range from 450 nm to 485 nm. For another example, the first radiation component 127 can be infrared radiation having a wavelength on the range from 800 nm to 900 nm, the second radiation component 128 can be red radiation having a wavelength on the range from 650 nm to 675 nm, the third radiation component 129 can be yellow radiation having a wavelength on the range from 575 nm to 580 nm, the fourth radiation component 130 can be green radiation having a wavelength on the range from 530 nm to 545 nm, and the fifth radiation component 131 can be blue radiation having a wavelength on the range from 460 nm to 475 nm.

As illustrated in the section view 130, the red and infrared radiation components 127 and 128 reach arteries 111 located in the hypodermis 114, the yellow radiation component 129 reach blood vessels located in a portion of the hypodermis 114 adjacent to the dermis 115, the green radiation component 130 reach arterioles 112 located in the dermis 115, and the blue radiation component 131 reach capillaries 112 located in a portion of the dermis 115 adjacent to the epidermis 116. Therefore, shorter wavelengths are reflected off smaller blood vessels, i.e. blood vessels nearer to a skin surface, than longer wavelengths. The photodetector 107 of the PPG sensor 101 is configured produce a measurement signal that comprises wavelength-specific component signals indicative of received wavelengths reflected off the blood vessels. FIG. 1b shows graphs illustrating exemplifying wavelength-specific component signals 117, 118, 119, 120, and 121 of an exemplifying measurement signal 122. The wavelength-specific component signal 117 corresponds to infrared radiation, the wavelength-specific component signal 118 corresponds to red radiation, the wavelength-specific component signal 119 corresponds to yellow radiation, the wavelength-specific component signal 120 corresponds to green radiation, and the wavelength-specific component signal 121 corresponds to blue radiation. The photodetector 107 may comprise for example many photodiodes or phototransistors that are sensitive to different wavelengths, or the photodetector 107 may comprise filters to implement wavelength separation.

The apparatus comprises a pressure instrument 102 configured to produce controllable mechanical pressure P applied on the blood vessels. The apparatus comprises a control system 103 configured to control the pressure instrument 102 to change, i.e. to decrease or increase, the mechanical pressure linearly with respect to time t when the electromagnetic radiation is emitted to the blood vessels and a reflected part of the electromagnetic radiation is received from the blood vessels. FIG. 1b shows a line 132 illustrating the time dependence of the mechanical pressure in the exemplifying situation in which the mechanical pressure has been linearly decreased and the above-mentioned wavelength-specific component signals 117-121 have been measured.

In the exemplifying apparatus illustrated in FIG. 1a, the pressure instrument 102 comprises a pressure sensor 109 for measuring the mechanical pressure P directed by the fingertip 108 to the pressure sensor 108 and pressing means for controllably pressing the fingertip 108 against the PPG sensor 101 and the pressure sensor 109. In this exemplifying apparatus, the pressing means comprise a pressing element 105 and a force generator 104 for directing force to the pressing element 105. The force generator 104 may comprise for example an electric stepper motor and a threaded rod or some other suitable elements for generating force.

The control system 103 is configured to find, from each of the wavelength-specific component signals 117-121, a portion whose envelope has exponential change, i.e. exponential growth or exponential decrease, with respect to time when the mechanical pressure decreases linearly with respect to time. The control system 103 is configured to produce, for each of the wavelength-specific component signals 117-121, an estimate for a coefficient of time related to the above-mentioned exponential change. The coefficient of time is indicative of the compliance of the blood vessels reflecting off the wavelength corresponding to the wavelength-specific component signal under consideration and thereby also the stiffness of these blood vessels.

As mentioned above, in the exemplifying case illustrated in FIG. 1b, the mechanical pressure is linearly decreased and thus each of the wavelength-specific component signals 117-121 has a portion whose envelope has exponential growth. FIG. 1b shows a magnification of a part of the wavelength-specific component signal 119. In FIG. 1b, the envelopes of the wavelength-specific component signals 117-121 are depicted with thick lines. The exponential growth ˜e^{αYt} on a part of the envelope of the wavelength-specific component signal 119 is depicted with a dashed line. In this case, the coefficient of time related to the exponential growth is ay. Thus, the coefficient of time ay is indicative of the compliance of the blood vessels from which yellow light is reflected off and thereby also the stiffness of the blood vessels from which the yellow light is reflected off.

There are many ways to find the portion whose envelope has the exponential change when the mechanical pressure changes linearly and to produce the estimate for the coefficient of time related to the exponential change. For example, curve fitting based on e.g. the least-mean-square “LMS” method can be used. Thus, apparatuses according to embodiments of the invention are not limited to any specific ways to find the portion whose envelope has the exponential change and to produce the estimate for the coefficient of time related to the exponential change. For example, in an apparatus according to an exemplifying and non-limiting embodiment, the control system 103 is configured to convert the wavelength-specific components 117-121 of the measurement signal to a logarithmic scale. FIG. 1c shows graphs illustrating the converted wavelength-specific component signals 117′, 118′, 119′, 120′, and 121′ which corresponds to the wavelength-specific component signals 117, 118, 119, 120, and 121 shown in FIG. 1b, respectively. Due to the logarithmic conversion, the exponential change is converted into linear change that is depicted with lines 117″, 118″, 119″, 120″, and 121″ shown in FIG. 1c. The control system 103 is configured to estimate the slope of the linear change of the envelope of each converted wavelength-specific component signal. The slope is the coefficient of time related to the exponential change. In the exemplifying case shown in FIGS. 1b and 1c, the slope i.e. the coefficient of time related to the infrared light is α_{IR}, the coefficient of time related to the red light is α_{R}, the coefficient of time related to the yellow light is ay, the coefficient of time related to the green light is α_{G}, and the coefficient of time related to the blue light is α_{B}. Thus, for example, α_{R} is the indicative of compliance of arteries and thereby also the stiffness of the arteries, α_{G} is indicative of compliance of arterioles and thereby also the stiffness of the arterioles, and α_{B} is indicative of compliance of capillaries and thereby also the stiffness of the capillaries.

The control system 103 is configured to optimize resistance values of a circuit model to minimize differences between waveforms of node voltages of the circuit model and measured waveforms of blood pressures prevailing in the blood vessels reflecting off different ones of the wavelengths. Capacitor values of the circuit model are based on the above-mentioned compliances of the blood vessels, and the optimized resistance values are indicative of the flow resistances of the blood vessels. FIG. 1d shows an exemplifying circuit model used for estimating the flow resistances of blood vessels. In this exemplifying case, the circuit model comprises three circuit segments 150, 151, and 152. The circuit segment 150 may model for example arteries located in the hypodermis, the circuit segment 151 may model for example arterioles located in the dermis, and the circuit segment 152 may model for example capillaries located in an upper portion of the dermis. Node voltage U1 can model the blood pressure prevailing in the blood vessels reflecting off infrared and red light, node voltage U2 can model the blood pressure prevailing in the blood vessels reflecting off green light, and node voltage U3 can model the blood pressure prevailing in the blood vessels reflecting off blue light. Node voltage U0 can have a waveform whose maximum is measured systolic blood pressure and whose minimum is measured diastolic blood pressure. The shape of the waveform of the node voltage U0 can be modelled e.g. as U0=V1_AC+V2_AC+V1_DC, where V1_AC=V1 sin(2π×HR/60×t) and V2_AC=V2 sin(2π×2HR/60×t), where t is time in seconds, HR is a heart rate as heart beats per minute, and V1, V2, and V1_DC are selected so that the maximum of U0 is the measured systolic blood pressure and the minimum of U0 is the measured diastolic blood pressure. Voltage levels in the circuit model are advantageously selected so that volts correspond directly to millimeters of mercury “mmHg”. In an exemplifying case, HR=60/min, V1=10 volts, V2=10 volts, and V1_DC=from 80 to 120 volts, e.g. 100 volts. In this exemplifying case, the peak-to-peak of U0 is from 25 to 40 volts e.g. about 35 V. Voltage V2_DC models the blood pressure after the capillaries and it can be on the range from 0 to 10 volts, or on the range from 3 to 8 volts. As mentioned above, the capacitor values of the circuit model are based on the above-mentioned compliances of the blood vessels. In this exemplifying case, the capacitor values of the circuit model can be for example: C1,1=C1,2=α_{IR}/2 or α_{R}/2, C2,1=C2,2=α_{G}/2, and C3=α_{B}.

It is to be noted that the above-described way to model the input voltage U0 of the circuit model is an example only. It is also possible to measure or estimate a waveform of blood pressure at the beginning of the greatest blood vessels, e.g. arteries, modelled by the circuit model and thereafter use the measured or estimated blood pressure waveform as the input voltage U0 of the circuit model. Furthermore, characteristic values e.g. amplitudes and phases of different sinusoidal AC components and a DC component of the input voltage U0 can be optimized parameters along with the resistance values of the circuit model. Furthermore, instantaneous values forming the waveform the input voltage U0 can be optimized parameters along with the resistance values of the circuit model. Thus, the invention is not limited to any specific ways to obtain the input voltage U0 of the circuit model. The measurements are advantageously calibrated so that e.g. the measured pressure values correspond directly to voltage values of the circuit model.

In an apparatus according to an exemplifying and non-limiting embodiment, the control system 103 is configured to variate the resistance values R1,1, R1,2, R2,1, R2,2, R3,1, and R3,2 as long as a sum of time integrals of squares of differences between the waveforms of the node voltages U1, U2, and U3 and the measured waveforms of the blood pressures is decreasing. The optimized resistance values are indicative of the flow resistances of the blood vessels reflecting off different wavelengths. In the above-described exemplifying case, R1,1+R1,2 is the flow resistance of the arteries, R2,1+R2,2 is the flow resistance of the arterioles, and R3,1+R3,2 is the flow resistance of the capillaries. The resistance values can be varied e.g. stochastically in which case the optimization process is a simulated evolution process. It is to be noted that any suitable multivariable optimization technique can be used, and the invention is not limited to any specific multivariable optimization techniques. Furthermore, it is to be noted that the circuit model shown in FIG. 1d is a non-limiting example only, and different circuit models can be used in conjunction with different embodiments of the invention. For example, blood vessels reflecting off a given wavelength, such as e.g. arteries, can be modelled with only one resistor-capacitor “RC” loop or with three or more subsequently connected RC-loops.

The waveforms of the blood pressures prevailing in the blood vessels reflecting off different wavelengths can be measured for example in the following way: The maximum value of blood pressure prevailing in blood vessels reflecting off a given wavelength is a value of a down ramping pressure, e.g. the pressure 132 in FIG. 1b, at which the corresponding wavelength-specific component signal 117, 118, 119, 120, or 121 has 50% of its maximum peak-to-peak value. For example, in the exemplifying case shown in FIG. 1b, the maximum value of the blood pressure prevailing in blood vessels reflecting off infrared and red light is P1, the maximum value of the blood pressure prevailing in blood vessels reflecting off yellow light is P2, and the maximum value of the blood pressure prevailing in blood vessels reflecting off green light is P3. The pressure values P1, P2, and P3 can be e.g. 127 mmHg, 100 mmHg, and 81 mmHg, respectively. The mean value of blood pressure prevailing in blood vessels reflecting off a given wavelength is a value of the down ramping pressure 132 at which the corresponding wavelength-specific component signal 117, 118, 119, 120, or 121 has its maximum peak-to-peak value. The shape of the waveform of blood pressure prevailing in blood vessels reflecting off a given wavelength is the shape of the corresponding wavelength-specific component signal 117, 118, 119, 120, or 121 when the mechanical pressure is kept constant, advantageously zero. The waveform of each wavelength-specific component signal can then be scaled and shifted so that its maximum value is the above-mentioned maximum value and its mean value is the above-mentioned mean value. More information about ways to measure the waveforms of blood pressures prevailing in blood vessels reflecting off different wavelengths can be found e.g. from the publication Panula, T., et al.: “An instrument for measuring blood pressure and assessing cardiovascular health from the fingertip”, Biosensors and Bioelectronics, Volume 167, 1 Nov. 2020, 112483. It is to be noted that the invention is not limited to any specific way to measure the waveforms of the blood pressures prevailing in the blood vessels reflecting off different wavelengths.

In the exemplifying apparatus illustrated in FIG. 1a, the PPG sensor 101 is configured to emit and receive different wavelengths simultaneously. It is however also possible that the control system of an apparatus according to an exemplifying and non-limiting embodiment is configured to control the PPG sensor to variate the wavelength of the electromagnetic radiation, and to produce successively the coefficients of time, such as αIR, α_{R}, α_{Y}, α_{G}, and α_{B}, for different wavelengths as well as the waveforms of the blood pressures prevailing in the blood vessels reflecting off different wavelengths.

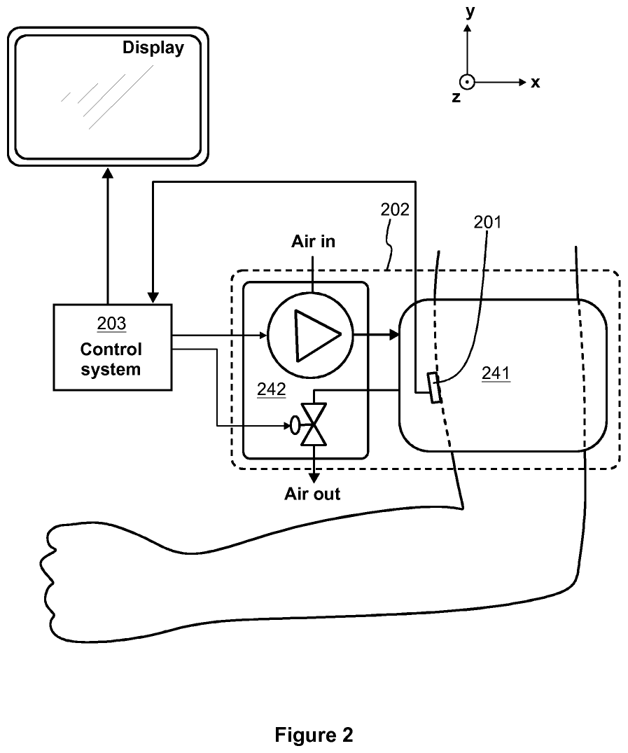

FIG. 2 shows a schematic illustration of an apparatus according to an exemplifying and non-limiting embodiment for measuring flow resistances of blood vessels. The apparatus comprises a photoplethysmography “PPG” sensor 201 for emitting electromagnetic radiation having different wavelengths to blood vessels of an arm and for receiving a part of the electromagnetic radiation reflected off the blood vessels. The PPG sensor 201 is configured to produce a measurement signal indicative of the received part of the electromagnetic radiation so that the measurement signal comprises wavelength-specific component signals indicative of received wavelengths reflected off the blood vessels, where a shorter one of the wavelengths is reflected off smaller ones of the blood vessels than a longer one of the wavelengths.

The apparatus comprises a pressure instrument 202 configured to produce controllable mechanical pressure applied on the blood vessels. The apparatus comprises a control system 203 configured to control the pressure instrument 202 to decrease the mechanical pressure linearly with respect to time when the electromagnetic radiation is emitted to the blood vessels and the reflected electromagnetic radiation is received from the blood vessels. The control system 203 is configured to find, from each wavelength-specific component signal, a portion whose envelope has exponential change with respect to time when the mechanical pressure changes linearly with respect to time and to produce, for each wavelength-specific component signal, an estimate for a coefficient of time related to the exponential change. The coefficient of time is indicative of the compliance of the blood vessels reflecting off the wavelength under consideration, and thereby the coefficient of time is indicative of the stiffness of the blood vessels. The control system 203 is configured to optimize resistance values of a circuit model to minimize differences between waveforms of node voltages of the circuit model and measured waveforms of blood pressures being in the blood vessels reflecting off different ones of the wavelengths. Capacitor values of the circuit model are based on the above-mentioned compliances of the blood vessels, and the optimized resistance values are indicative of the flow resistances of the blood vessels.

In the exemplifying apparatus illustrated in FIG. 2, the pressure instrument 202 comprises a cuff and 241 a pump system 242 configured to control gas pressure inside the cuff and thereby to control the mechanical pressure directed to the blood vessels when the PPG sensor 201 emits and receives the electromagnetic radiation to and from the arm. The PPG sensor is located on an inner surface of the cuff.

Each of the control systems 103 and 203 shown in FIGS. 1a and 2 can be implemented for example with one or more processor circuits, each of which can be a programmable processor circuit provided with appropriate software, a dedicated hardware processor such as for example an application specific integrated circuit “ASIC”, or a configurable hardware processor such as for example a field programmable gate array “FPGA”. Each of the control systems 103 and 203 may further comprise memory implemented for example with one or more memory circuits each of which can be e.g. a random-access memory “RAM” device.

An apparatus according to an exemplifying and non-limiting embodiment for measuring flow resistances of blood vessels comprises a control system configured to:
a) estimate cardiac output “CO” according to the formula CO=stroke volume “SV”×Heart rate “HR”×α_{C}×(√(R_{cr}(0))+Π(−R_{cr}(τₘᵢₙ))), where R_{cr} is the autocorrelation of the arterial blood pressure waveform, τₘᵢₙ is the smallest time shift in the autocorrelation that gives a peak in the autocorrelation which is comparable to the value R_{cr}(0) at zero shift in the autocorrelation, and ac is a calibration constant that can be determined e.g. experimentally,
b) estimate the mean arterial pressure “MAP” with a suitable known method,
c) compute systemic vascular resistance “SVR”=MAP/CO,
d) compute the blood flow through the vessels F=blood pressure at arteries/SVR, and
e) compute the flow resistance R at a given point in the vasculature, e.g. arteries, arterioles, or capillaries=the blood pressure at the given point/F.

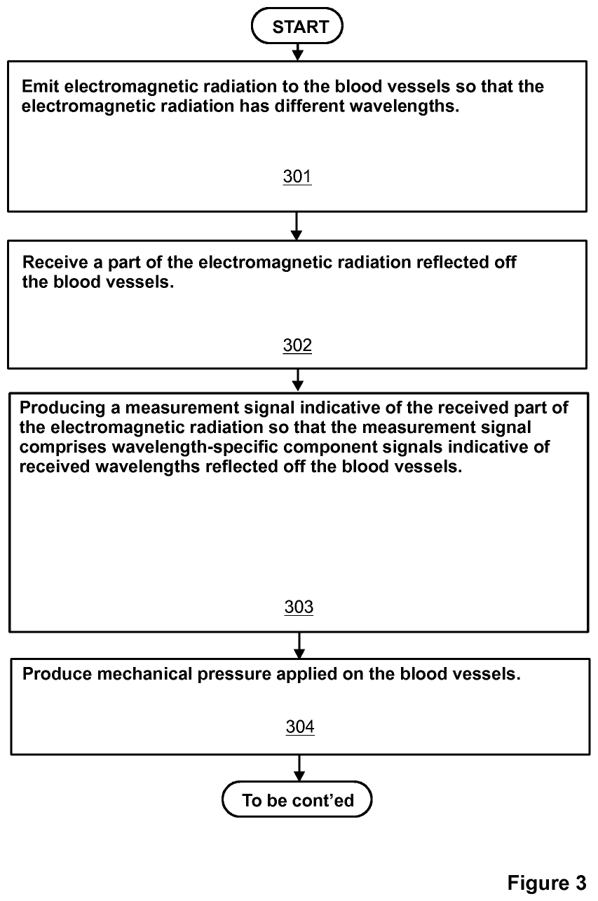

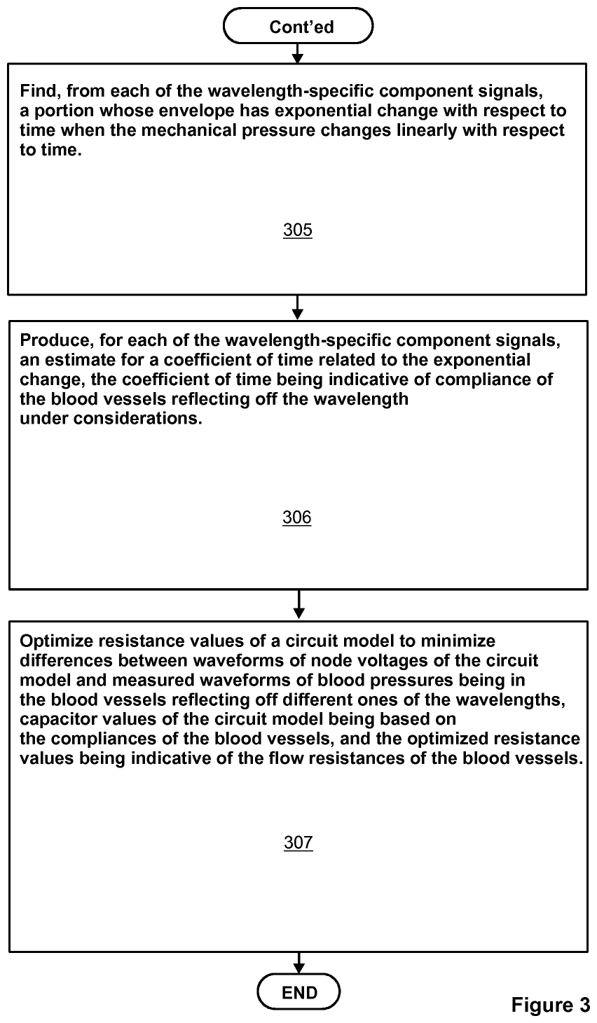

FIG. 3 shows a flowchart of a method according to an exemplifying and non-limiting embodiment for measuring flow resistances of blood vessels. The method comprises the following actions:
action 301: emitting electromagnetic radiation to the blood vessels so that the electromagnetic radiation has different wavelengths,
action 302: receiving a part of the electromagnetic radiation reflected off the blood vessels,
action 303: producing a measurement signal indicative of the received part of the electromagnetic radiation so that the measurement signal comprises wavelength-specific component signals indicative of received wavelengths reflected off the blood vessels, where a shorter one of the wavelengths is reflected off smaller ones of the blood vessels than a longer one of the wavelengths,
action 304: producing mechanical pressure applied on the blood vessels,
action 305: finding, from each of the wavelength-specific component signals, a portion whose envelope has exponential change with respect to time when the mechanical pressure changes linearly with respect to time,
action 306: producing, for each of the wavelength-specific component signals, an estimate for a coefficient of time related to the exponential change, the coefficient of time being indicative of compliance of the blood vessels reflecting off the wavelength under consideration, and
action 307: optimizing resistance values of a circuit model to minimize differences between waveforms of node voltages of the circuit model and measured waveforms of blood pressures prevailing in the blood vessels reflecting off different ones of the wavelengths, wherein capacitor values of the circuit model are based on the compliances of the blood vessels, and the optimized resistance values are indicative of the flow resistances of the blood vessels.

In a method according to an exemplifying and non-limiting embodiment, the electromagnetic radiation has wavelengths selected from the following ranges: from 625 nm to 1000 nm, from 565 nm to 590 nm, from 500 nm to 565 nm, and from 450 nm to 485 nm.

A method according to an exemplifying and non-limiting embodiment comprises converting the measurement signal to a logarithmic scale, finding from the converted measurement signal a portion whose envelope has linear change with respect to time, and producing an estimate for a slope of the envelope of the converted measurement signal related to the linear change. The slope of the linear change is the coefficient of time related to the exponential change.

In a method according to an exemplifying and non-limiting embodiment, the mechanical pressure is directed to a fingertip or a toe of an individual.

In a method according to an exemplifying and non-limiting embodiment, the mechanical pressure is directed to an arm of an individual with a cuff and a pump system configured to control gas pressure inside the cuff. In this exemplifying case, the photoplethysmography sensor is located on an inner surface of the cuff.

In a method according to an exemplifying and non-limiting embodiment, the resistance values of the circuit model are optimized so that the resistance values are variated to as long as a sum of time integrals of squares of differences between the waveforms of the node voltages and the measured waveforms of the blood pressures is decreasing.

A method according to an exemplifying and non-limiting embodiment for measuring flow resistances of blood vessels comprises the following:
a) estimating cardiac output “CO” according to the formula CO=stroke volume “SV”×Heart rate “HR”×α_{C}×(√(R_{cr}(0))+√(−R_{cr}(τₘᵢₙ))), where R_{cr} is the autocorrelation of the arterial blood pressure waveform, τₘᵢₙ is the smallest time shift in the autocorrelation that gives a peak in the autocorrelation which is comparable to the value R_{cr}(0) at zero shift in the autocorrelation, and ac is a calibration constant that can be determined e.g. experimentally,
b) estimating the mean arterial pressure “MAP” with a suitable known method,
c) computing systemic vascular resistance “SVR”=MAP/CO,
d) computing the blood flow through the vessels F=blood pressure at arteries/SVR, and
e) computing the flow resistance R at a given point in the vasculature, e.g. arteries, arterioles, or capillaries=the blood pressure at the given point/F.

A computer program according to an exemplifying and non-limiting embodiment comprises computer executable instructions for controlling a programmable processing system to carry out actions related to a method according to any of the above-described exemplifying and non-limiting embodiments.

A computer program according to an exemplifying and non-limiting embodiment comprises software modules for measuring flow resistances of blood vessels. The software modules comprise computer executable instructions for controlling a programmable processing system to:
control a photoplethysmography sensor to emit electromagnetic radiation to the blood vessels, to receive a part of the electromagnetic radiation reflected off the blood vessels, and to produce a measurement signal indicative of the received part of the electromagnetic radiation so that the electromagnetic radiation has different wavelengths and the measurement signal comprises wavelength-specific component signals indicative of received wavelengths reflected off the blood vessels, where a shorter one of the wavelengths is reflected off smaller ones of the blood vessels than a longer one of the wavelengths,
control a pressure instrument to produce mechanical pressure applied on the blood vessels,
control the programmable processing system to find, from each of the wavelength-specific component signals, a portion whose envelope has exponential change with respect to time when the mechanical pressure changes linearly with respect to time, and produce, for each of the wavelength-specific component signals, an estimate for a coefficient of time related to the exponential change, the coefficient of time being indicative of compliance of the blood vessels reflecting off the wavelength under consideration, and
optimize resistance values of a circuit model to minimize differences between waveforms of node voltages of the circuit model and measured waveforms of blood pressures being in the blood vessels reflecting off different ones of the wavelengths, wherein capacitor values of the circuit model are based on the compliances of the blood vessels and the optimized resistance values are indicative of the flow resistances of the blood vessels.

The software modules can be for example subroutines or functions implemented with programming tools suitable for the programmable processing system.

A computer program product according to an exemplifying and non-limiting embodiment comprises a computer readable medium, e.g. a compact disc “CD”, encoded with a computer program according to an exemplifying embodiment.

A signal according to an exemplifying and non-limiting embodiment is encoded to carry information defining a computer program according to an exemplifying embodiment.

A computer program according to an exemplifying and non-limiting embodiment may constitute e.g. a part of a software of a mobile device, e.g. a smart phone or a wearable device.

A computer program according to an exemplifying and non-limiting embodiment comprises computer executable instructions for controlling a programmable processing system to:
a) estimate cardiac output “CO” according to the formula CO=stroke volume “SV”×Heart rate “HR”×α_{C}×(√(R_{cr}(0))+√(−R_{cr}(τₘᵢₙ))), where R_{cr} is the autocorrelation of the arterial blood pressure waveform, τₘᵢₙ is the smallest time shift in the autocorrelation that gives a peak in the autocorrelation which is comparable to the value R_{cr}(0) at zero shift in the autocorrelation, and ac is a calibration constant that can be determined e.g. experimentally,
b) compute systemic vascular resistance “SVR”=MAP/CO, where MAP is the mean arterial pressure determined with a suitable known method,
c) compute the blood flow through the vessels F=blood pressure at arteries/SVR, and
d) compute the flow resistance R at a given point in the vasculature, e.g. arteries, arterioles, or capillaries=the blood pressure at the given point/F.

The specific examples provided in the description given above should not be construed as limiting the scope and/or the applicability of the appended claims. Lists and groups of examples provided in the description given above are not exhaustive unless otherwise explicitly stated.

1. An apparatus for measuring flow resistances of blood vessels, the apparatus comprising:
a photoplethysmography sensor configured to emit electromagnetic radiation to the blood vessels, to receive a part of the electromagnetic radiation reflected off the blood vessels, and to produce a measurement signal indicative of the received part of the electromagnetic radiation so that the electromagnetic radiation has different wavelengths and the measurement signal comprises wavelength-specific component signals indicative of received wavelengths reflected off the blood vessels, where a shorter one of the wavelengths is reflected off smaller ones of the blood vessels than a longer one of the wavelengths,
a pressure instrument configured to produce controllable mechanical pressure applied on the blood vessels, and
a control system configured to find, from each of the wavelength-specific component signals, a portion whose envelope has exponential change with respect to time when the mechanical pressure changes linearly with respect to time, and produce, for each of the wavelength-specific component signals, an estimate for a coefficient of time related to the exponential change, the coefficient of time being indicative of compliance of the blood vessels reflecting off the wavelength under consideration,
wherein the control system is configured to optimize resistance values of a circuit model to minimize differences between waveforms of node voltages of the circuit model and measured waveforms of blood pressures being in the blood vessels reflecting off different ones of the wavelengths, wherein capacitor values of the circuit model are based on the compliances of the blood vessels and the optimized resistance values are indicative of the flow resistances of the blood vessels.

2. The apparatus according to claim 1, wherein the photoplethysmography sensor is configured to emit, to the blood vessels, the electromagnetic radiation so that the electromagnetic radiation has the wavelengths selected from the following ranges: from 625 nm to 1000 nm, from 565 nm to 590 nm, from 500 nm to 565 nm, and from 450 nm to 485 nm.

3. The apparatus according to claim 1, wherein the control system is configured to convert the wavelength-specific component signals to a logarithmic scale, to find from each converted wavelength-specific component signal a portion whose envelope has linear change with respect to time, and to produce an estimate for a slope of the envelope of the converted wavelength-specific component signal related to the linear change, the slope being the coefficient of time related to the exponential change of the wavelength-specific component signal under consideration.

4. The apparatus according to claim 1, wherein the pressure instrument comprises a force generator and a pressing element configured to direct the mechanical pressure to a fingertip or a toe in accordance with a control signal generated by the control system.

5. The apparatus according to claim 1, wherein the pressure instrument comprises a cuff and a pump system configured to control gas pressure inside the cuff to direct the mechanical pressure to an arm.

6. The apparatus according to claim 1, wherein the control system is configured to variate the resistance values to as long as a sum of time integrals of squares of differences between the waveforms of the node voltages and the measured waveforms of blood pressures is decreasing.

7. A method for measuring flow resistances of blood vessels, the method comprising:
emitting electromagnetic radiation to the blood vessels so that the electromagnetic radiation has different wavelengths,
receiving a part of the electromagnetic radiation reflected off the blood vessels,
producing a measurement signal indicative of the received part of the electromagnetic radiation so that the measurement signal comprises wavelength-specific component signals indicative of received wavelengths reflected off the blood vessels, where a shorter one of the wavelengths is reflected off smaller ones of the blood vessels than a longer one of the wavelengths,
producing mechanical pressure applied on the blood vessels,
finding, from each of the wavelength-specific component signals, a portion whose envelope has exponential change with respect to time when the mechanical pressure changes linearly with respect to time,
producing, for each of the wavelength-specific component signals, an estimate for a coefficient of time related to the exponential change, the coefficient of time being indicative of compliance of the blood vessels reflecting off the wavelength under consideration, and
optimizing resistance values of a circuit model to minimize differences between waveforms of node voltages of the circuit model and measured waveforms of blood pressures being in the blood vessels reflecting off different ones of the wavelengths, wherein capacitor values of the circuit model are based on the compliances of the blood vessels and the optimized resistance values are indicative of the flow resistances of the blood vessels.

8. The method according to claim 7, wherein the electromagnetic radiation has wavelengths selected from the following ranges: from 625 nm to 1000 nm, from 565 nm to 590 nm, from 500 nm to 565 nm, and from 450 nm to 485 nm.

9. The method according to claim 7, wherein the method comprises converting the wavelength-specific component signals to a logarithmic scale, finding from each converted wavelength-specific component signal a portion whose envelope has linear change with respect to time, and producing an estimate for a slope of the envelope of the converted wavelength-specific component signal related to the linear change, the slope being the coefficient of time related to the exponential change of the wavelength-specific component signal under consideration.

10. The method according to claim 7, wherein the mechanical pressure is directed to a fingertip or a toe.

11. The method according to claim 7, wherein the method comprises variating the resistance values to as long as a sum of time integrals of squares of differences between the waveforms of the node voltages and the measured waveforms of the blood pressures is decreasing.

12. A computer program product comprising a non-transitory computer readable medium encoded with a computer program for measuring flow resistances of blood vessels, the computer program comprising computer executable instructions that, when executed by a programmable processing system, cause the programmable processing system to:
control a photoplethysmography sensor to emit electromagnetic radiation to the blood vessels, to receive a part of the electromagnetic radiation reflected off the blood vessels, and to produce a measurement signal indicative of the received part of the electromagnetic radiation so that the electromagnetic radiation has different wavelengths and the measurement signal comprises wavelength-specific component signals indicative of received wavelengths reflected off the blood vessels, where a shorter one of the wavelengths is reflected off smaller ones of the blood vessels than a longer one of the wavelengths,
control a pressure instrument to produce mechanical pressure applied on the blood vessels,
control the programmable processing system to find, from each of the wavelength-specific component signals, a portion whose envelope has exponential change with respect to time when the mechanical pressure changes linearly with respect to time, and produce, for each of the wavelength-specific component signals, an estimate for a coefficient of time related to the exponential change, the coefficient of time being indicative of compliance of the blood vessels reflecting off the wavelength under consideration, and
optimize resistance values of a circuit model to minimize differences between waveforms of node voltages of the circuit model and measured waveforms of blood pressures being in the blood vessels reflecting off different ones of the wavelengths, wherein capacitor values of the circuit model are based on the compliances of the blood vessels and the optimized resistance values are indicative of the flow resistances of the blood vessels.

13. (canceled)

14. The apparatus according to claim 2, wherein the control system is configured to convert the wavelength-specific component signals to a logarithmic scale, to find from each converted wavelength-specific component signal a portion whose envelope has linear change with respect to time, and to produce an estimate for a slope of the envelope of the converted wavelength-specific component signal related to the linear change, the slope being the coefficient of time related to the exponential change of the wavelength-specific component signal under consideration.

15. The apparatus according to claim 2, wherein the pressure instrument comprises a force generator and a pressing element configured to direct the mechanical pressure to a fingertip or a toe in accordance with a control signal generated by the control system.

16. The apparatus according to claim 3, wherein the pressure instrument comprises a force generator and a pressing element configured to direct the mechanical pressure to a fingertip or a toe in accordance with a control signal generated by the control system.

17. The apparatus according to claim 2, wherein the pressure instrument comprises a cuff and a pump system configured to control gas pressure inside the cuff to direct the mechanical pressure to an arm.

18. The apparatus according to claim 3, wherein the pressure instrument comprises a cuff and a pump system configured to control gas pressure inside the cuff to direct the mechanical pressure to an arm.

19. The apparatus according to claim 2, wherein the control system is configured to variate the resistance values to as long as a sum of time integrals of squares of differences between the waveforms of the node voltages and the measured waveforms of blood pressures is decreasing.

20. The apparatus according to claim 3, wherein the control system is configured to variate the resistance values to as long as a sum of time integrals of squares of differences between the waveforms of the node voltages and the measured waveforms of blood pressures is decreasing.

21. The apparatus according to claim 4, wherein the control system is configured to variate the resistance values to as long as a sum of time integrals of squares of differences between the waveforms of the node voltages and the measured waveforms of blood pressures is decreasing.
